(19) United States
(12) Patent Application Publication
(10) Pub. No.: US 2022/0389510 A1
(43) Pub. Date: Dec. 8, 2022

(54) SYSTEMS AND METHODS FOR MULTIMODAL ANALYSIS OF SURGICAL DRAIN FLUID USING INTERCHANGEABLE AND CUSTOMIZABLE NUCLEIC ACID BASED TESTS

(71) Applicant: Washington University, St. Louis, MO (US)

(72) Inventors: Jose Zevallos, St. Louis, MO (US); Aadel Chaudhuri, St. Louis, MO (US)

(73) Assignee: Washington University, St. Louis, MO (US)

(21) Appl. No.: 17/831,103

(22) Filed: Jun. 2, 2022

Related U.S. Application Data
(60) Provisional application No. 63/195,871, filed on Jun. 2, 2021.

(51) Int. Cl. C12Q 1/6883

(52) U.S. Cl. CPC C12Q 1/6883

(57) ABSTRACT

A method for detecting at least one surgery-related condition within a surgical site of a subject following a surgery is disclosed. The method includes providing at least one modular nucleic acid assay, obtaining a surgical drainage sample from the surgical site of the subject, isolating a nucleic acid-containing portion from the sample, and detecting and quantifying at least a portion of the amount of nucleic acids within the nucleic acid-containing portion using the at least one nucleic acid assay to produce at least one assay result. At least one assay result may be provided to a practitioner and used to select at least one additional treatment. The modular nucleic acid assays may be used in any combination with a single surgical drainage sample to assess minimal residual cancer, local immune environment, infection, transplant organ rejection, and/or free flap failure.

### CROSS-REFERENCE TO RELATED APPLICATIONS

This application claims priority from U.S. Provisional Application Ser. No. 63/195,871 filed on Jun. 2, 2021, the content of which is incorporated herein by reference in its entirety.

### STATEMENT REGARDING FEDERALLY SPONSORED RESEARCH OR DEVELOPMENT

This work was supported by the U.S. Department of Veterans Affairs, and the Federal Government has certain rights in this invention.

### FIELD OF THE DISCLOSURE

The present disclosure generally relates to systems and methods of monitoring a surgical wound of a patient to assess surgical outcomes and potential complications, as well as to guide the selection of follow-up treatments.

### BACKGROUND OF THE DISCLOSURE

For patients undergoing surgical treatments, such as resectioning surgery, dissection surgery, excision surgery, and transplantation surgery, it may be challenging to monitor the patient to assess the surgical outcome, to detect the development of complications associated with the surgery, and/or to select additional treatments. Depending on the extent of the surgery and condition of the patient, it may be inadvisable to subject the patient to any of the various medical imaging modalities, and these modalities may not provide sufficient information to assess at least some of the concerns of a medical practitioner regarding the post-surgical condition of the patient. Biomarkers isolated from blood samples of the patient may provide some information regarding the post-surgical patient's condition, but typically any biomarkers released from a surgical site are diluted by the blood volume and transported by blood flow within the circulatory vessels to regions distal to the surgical site. As a consequence, blood biomarkers may be present at very low concentrations and may require a highly sensitive assay, if indeed the concentrations are above an assay's lowest detectable concentration. In addition, biomarkers detected in a patient blood sample may originate from sources other than the surgical site.

Surgical drain fluid represents a potentially rich source of biomarkers indicative of a post-surgical patient's condition and prognosis. Surgical drain fluid originates at the surgery site and is drained from the patient without further dilution. Surgical drain fluid biomarkers are typically present at higher concentrations than corresponding blood concentrations. Further, biomarkers in the surgical drain fluid may have a higher range of concentrations and therefore may be detectable using assays with higher minimum detection limits.

### SUMMARY OF THE DISCLOSURE

In one aspect, a method for detecting at least one surgery-related condition within a surgical site of a subject following a surgery is disclosed. The method includes providing at least one modular nucleic acid assay from a plurality of modular nucleic acid assays, obtaining a sample from the subject, isolating a nucleic acid-containing portion from the sample, detecting and quantifying at least a portion of the amount of nucleic acids within the nucleic acid-containing portion using the at least one nucleic acid assay to produce at least one assay result, and providing the at least one assay result to a practitioner. The sample may be a surgical drainage sample from the surgical site. The at least one assay result is indicative of the at least one surgery-related condition in the subject. In some aspects, the surgery is selected from a resectioning surgery, a dissection surgery, an excision surgery, a transplant surgery, a reconstructive surgery, and any combination thereof. In some aspects, the nucleic acid-containing portion isolated from the sample includes cfDNA, RNA, exosomes, tumor cells, immune cells, bacterial nucleic acids, viral nucleic acids, and any combination thereof. In some aspects, the plurality of modular nucleic acid assays includes whole genome sequencing, next generation DNA sequencing, next generation RNA sequencing, PCR, Western blot targeted capture, multiplex PCR, methylation & 16S droplet PCR, and any combination thereof. In some aspects, each modular nucleic acid assay of the plurality of modular nucleic acid assays is selected from a surgical margin assay module configured to detect and quantify tumor-associated nucleic acids indicative of minimal residual cancer within the surgical site, an immune environment assay module configured to detect and quantify immune response-related nucleic acids indicative of a local immune environment within the surgical site, an infection assay module configured to detect and quantify infection-associated nucleic acids indicative of an infection within the surgical site, a transplant assay configured to detect and quantify transplant organ nucleic acids indicative of a rejection of a transplant organ by the subject, a reconstruction assay module configured to detect and quantify flap failure-related nucleic acids indicative of early microvascular free flap failure, necrosis, and any combination thereof. In some aspects, the plurality of modular nucleic acid assays comprises a combination of at least two modular nucleic acid assays. In some aspects, isolating the nucleic acid-containing portion from the sample includes filtering the sample, centrifuging the sample, contacting the sample with a chromatography medium, and any combination thereof. In some aspects, the method further includes selecting an additional treatment based on the at least one assay result. In some aspects, the additional treatment is selected from radiotherapy, chemotherapy, follow-up surgery, active surveillance with imaging, antibiotic therapy, antiviral therapy, and any combination thereof. In some aspects, obtaining the sample from the subject further includes capturing a surgical drainage from a drainage tube associated with the surgery. In some aspects, obtaining the sample from the subject further comprises capturing a surgical drainage from the drainage tube within about 24 hours of the surgery.

In another aspect, a method of selecting at least one additional treatment for a subject following a surgery is disclosed. The method includes providing at least one modular nucleic acid assay from a plurality of modular nucleic acid assays, obtaining a sample from the subject, isolating a nucleic acid-containing portion from the sample, detecting and quantifying at least a portion of the amount of nucleic acids within the nucleic acid-containing portion using the at least one nucleic acid assay to produce at least one assay result, and selecting the at least one additional treatment based on the at least one assay result. The at least one assay result is indicative of the at least one surgery-related condition in the subject. In some aspects, the additional treatment is selected from radiotherapy, chemotherapy, follow-up surgery, active surveillance with imaging, antibiotic therapy, antiviral therapy, and any combination thereof. In some aspects, the nucleic acid-containing portion isolated from the sample includes cfDNA, RNA, exosomes, tumor cells, immune cells, bacterial nucleic acids, viral nucleic acids, and any combination thereof. In some aspects, the plurality of modular nucleic acid assays includes whole genome sequencing, next generation DNA sequencing, next generation RNA sequencing, PCR, Western blot targeted capture, multiplex PCR, methylation & 16S droplet PCR, and any combination thereof. In some aspects, each modular nucleic acid assay of the plurality of modular nucleic acid assays is selected from a surgical margin assay module configured to detect and quantify tumor-associated nucleic acids indicative of minimal residual cancer within the surgical site, an immune environment assay module configured to detect and quantify immune response-related nucleic acids indicative of a local immune environment within the surgical site, an infection assay module configured to detect and quantify infection-associated nucleic acids indicative of an infection within the surgical site, a transplant assay configured to detect and quantify transplant organ nucleic acids indicative of a rejection of a transplant organ by the subject, a reconstruction assay module configured to detect and quantify flap failure-related nucleic acids indicative of early microvascular free flap failure, necrosis, and any combination thereof. In some aspects, the plurality of modular nucleic acid assays comprises a combination of at least two modular nucleic acid assays. In some aspects, isolating the nucleic acid-containing portion from the sample includes filtering the sample, centrifuging the sample, contacting the sample with a chromatography medium, and any combination thereof. In some aspects, the method further includes selecting an additional treatment based on the at least one assay result. In some aspects, obtaining the sample from the subject further includes capturing a surgical drainage from a drainage tube associated with the surgery. In some aspects, obtaining the sample from the subject further comprises capturing a surgical drainage from the drainage tube within about 24 hours of the surgery.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a flow chart illustrating the steps of a method for detecting at least one surgery-related condition within a surgical site of a subject following a surgery in accordance with one aspect of the disclosure.

FIG. 2 is a schematic diagram illustrating the isolation and preservation of a nucleic acid-containing portion of a sample.

There are shown in the drawings arrangements, which are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE INVENTION

In various aspects, systems and methods for comprehensively measuring multiple aspects of a surgical wound are disclosed. The measurements are obtained from a single drain fluid sample obtained using a single surgical drain device, and make use of a practitioner-configurable combination of modular assays as described herein. The modular assays and associated methods together form a comprehensive platform for using surgical fluid as a measure of multiple components of wound physiology including, but not limited to, residual cancer, risk of infection, local immune environment, and risk of solid organ transplant rejection. The disclosed systems and methods incorporates sequencing into drain fluid analysis to enhance the quality and quantity of patient information provided to clinicians to facilitate the efficient planning of a patient's course of care. Without being limited to any particular theory, the disclosed systems and methods implement multimodal sequencing performed on single cells so that the specificity and sensitivity to low concentrations of biomarkers in a complex sample are high.

In various aspects, the disclosed systems comprise a modular sequencing/diagnostics platform that uses a single drain sample to measure a host of parameters related to wound healing, cancer residuals, immunotherapy efficacy, infection, transplantation complications, and the like. Biomarkers representative of the above conditions are captured from circulation in the drain fluid and subjected to additional analysis including, but not limited to, multimodal sequencing and PCR analysis to monitor the efficacy of the surgical treatment, to identify complications arising from the surgical treatment, and/or to select one or more additional treatments as described herein. Non-limiting examples of biomarkers suitable for capture from surgical drain fluid and analysis using the disclosed systems and methods include cfDNA, tumor-associated RNA/DNA, circulating tumor cells, exosomes, and infection-associated DNA, and the like.

In some aspects, the disclosed systems and methods enable the collection, preservation, and quantification of tumor-associated cells or nucleic acids/cfDNA as early measures of residual cancer. In other aspects, the disclosed systems and methods enable the collection, preservation, and quantification of immune response-related cells and nucleic acids as measures of systemic or tumor immunity. In other aspects, the disclosed systems and methods enable the collection, preservation, and quantification of bacteria and/or virus-derived nucleic acids as early measures of wound infection as measures of wound infection. In other aspects, the disclosed systems and methods enable the collection, preservation, and quantification of transplant-derived nucleic acids as measures of transplant rejection. In other aspects, the disclosed systems and methods enable the collection, preservation, and quantification of flap failure-related nucleic acids as early measures of microvascular free flap failure and/or necrosis.

### I. Modular Assays

In various aspects, the modular assays include nucleic acid-related measures including, but not limited to, whole genome sequencing, next generation DNA sequencing, next generation RNA sequencing, PCR, Western blot targeted capture, multiplex PCR, methylation & 16S droplet PCR, and any combination thereof. The nucleic acids detected and quantified using the modular assays are associated with various sources related to surgical wounds, and are indicative of one or more aspects of the local wound environment. Non-limiting examples of nucleic acid sources related to surgical wounds include tumor cells, immune cells, bacterial cells, viral host cells, donor organ cells, microvascular cells, cell-free DNA (cfDNA), cell-free RNA (cfRNA), exosomes, and any combination thereof.

In various aspects, the nucleic acids that are quantified by the disclosed modular assays are indicative of a variety of different aspects of the local wound environment associated with a surgical procedure. In these aspects, the systems and methods disclosed herein are suitable for use in conjunction with a variety of different surgical procedures including, but not limited to, resectioning surgery, dissection surgery, excision surgery, transplant surgery, reconstructive surgery, and any other suitable surgery type without limitation. In various aspects, a practitioner or surgeon may select any combination of a plurality of modular assays without limitation. In some aspects, a practitioner or surgeon may order specific or customized assays tailored to a specific indication, surgery type, surgery site, or any other criterion without limitation.

In various aspects, the disclosed modular assays are configured for use with surgical drain fluid. In some aspects, each modular assay may analyze a separately obtained surgical drain fluid sample. In other aspects, a single surgical fluid sample may be obtained and subjected to analysis by multiple modular assays. In other additional aspects, a single surgical fluid sample may be obtained and analyzed by all selected modular assays.

In some aspects, at least a portion of the modular assays may be based on corresponding blood, plasma, urine, or other fluid sample assays that are modified to render the assay compatible with surgical drain fluid samples. Non-limiting examples of corresponding assays suitable for modification for use with surgical drain fluid samples include liquid biopsy assays such as NavDx™ (Naveris, Natick, Mass., USA) used for the detection of circulating tumor DNA (ctDNA) and any other suitable corresponding assay without limitation. In other aspects, at least a portion of the modular assays may be developed de novo for use with the surgical drain fluid sample.

Non-limiting examples of suitable assays that may be modified to produce one or more of the modular assays suitable for the analysis of the surgical drain fluid samples as described herein are described in Molecular Diagnosis and Therapy (2021) 25:757-774, the content of which is incorporated by reference in its entirety.

a) Surgical Margin Assay Module

In some aspects, the modular assays may include a surgical margin assay module configured to detect and quantify tumor-associated nucleic acids or other genetic material indicative of minimal residual cancer within the surgical site. The surgical margin assay module provides a proximal measure of tumor cells remaining in the surgical region that may serve as a molecular surgical margin proximal measure of tumor cells remaining in the surgical region and may further provide information useful in the selection of additional post-operative therapies.

In various aspects, the tumor-associated genetic material detected and quantified using the surgical margin assay module includes, but is not limited to, cell-free DNA, RNA, proteins, exosomes, and any combination thereof. In other aspects, the tumor-associated genetic material is produced by or associated with a plurality of cancer cells. Non-limiting examples of tumor-associated genetic materials include mutations, overexpression, and underexpression of genes associated with cancer cells. Non-limiting examples of cancer cells include oropharyngeal cancer cells, lung cancer cells, breast cancer cells, melanoma cells, colon cancer cells, thyroid cancer cells, prostate cancer cells, ovarian cancer cells, testicular cancer cells, penile cancer cells, cervical cancer cells, anal cancer cells, brain cancer cells, liver cancer cells, pancreatic cancer cells, and testicular cancer cells.

Additional non-limiting examples of cancer cells include cells from a variety of cancer types including Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

In other aspects, the surgical margin assay module may be further configured to detect and quantify additional genetic material indicative of a prognosis or a recommended additional post-surgical treatment. By way of non-limiting example, the surgical margin assay module may measure HPV DNA within drain fluid samples collected following neck dissection surgery. The presence or absence of residual HPV DNA after surgery may be used as a proximal liquid biomarker to guide the selection of postoperative radiation therapy or chemotherapy in the setting of treatment de-intensification.

b) Immune Environment Assay Module

In some aspects, the modular assays may include an immune environment assay module configured to detect and quantify immune response-related nucleic acids indicative of a local immune environment within the surgical site. In various aspects, the immune response-related genetic material detected and quantified using the immune environment assay module includes, but is not limited to, cell-free DNA, RNA, proteins, exosomes, and any combination thereof. In other aspects, the immune response-related genetic material is produced by or associated with a plurality of immune cells within the surgical region. Non-limiting examples of immune response-related genetic material include genes encoding cellular markers associated with immune cells, overexpression or underexpression of genes encoding cytokines or other molecules indicative of an immune environment. The immune environment assay module provides a proximal measure of immune cell activity within the surgical region that may serve to define a prognosis, to characterize the immune environment within the surgical region, or to provide information useful in the evaluation of a patient's response to immunotherapy.

c) Infection Assay Module

In some aspects, the modular assays may include an infection assay module configured to detect and quantify infection-associated nucleic acids indicative of an infection within the surgical site. In various aspects, the infection-associated genetic material detected and quantified using the infection assay module includes, but is not limited to, microbe nucleic acids as early markers of wound or surgical site infection or fistula. In various aspects, the infection assay module may perform whole genome sequencing of bacteria, fungi, and viruses, as well as targeted capture, multiplex PCR, methylation & 16S droplet PCR. The results of the infection assay module may be used to select appropriately targeted antibiotic compositions and doses more quickly and precisely than wound culture. In other aspects, serial results of the infection assay module may be used to monitor the efficacy of antibiotic treatment.

d) Transplant Assay Module

In some aspects, the modular assays may include a transplant assay module configured to detect and quantify transplant organ nucleic acids indicative of a rejection of a transplant organ by the subject. In various aspects, transplant organ nucleic acids detected and quantified using the transplant assay module include cfDNA produced by the donor organ. In additional aspects, the transplant assay module may detect overexpression or underexpression of genes encoding biomarkers such as cytokines indicative of acute or chronic transplant organ rejection.

e) Reconstruction Assay Module

In some aspects, the modular assays may include a reconstruction assay module configured to detect and quantify flap failure-related nucleic acids indicative of early microvascular free flap failure, necrosis, and any combination thereof. In various aspects, flap failure-related nucleic acids detected and quantified using the reconstruction assay module include genes encoding biomarkers for early microvascular free flap failure or necrosis. Non-limiting examples of suitable biomarkers for early microvascular free flap failure or necrosis include cfDNA concentrations of the genes Prol1, Muc1, Fcnb, Il1b, and/or Vcsa1.

f) Customized Assay Modules

In various aspects, the modular assays may include customized assay modules as specified by a practitioner based on the type of surgery and/or surgery-related condition to be monitored. In some aspects, the customized assay module may be selected to perform analyses of surgical drain fluid samples in addition to the analyses performed by the modular assays described above, or as a substitute for the analyses performed by the modular assays described above.

The tumor-associated genetic material may be isolated from the sample using any suitable method without limitation. Non-limiting examples of suitable isolation methods include filtering the sample, centrifuging the sample, contacting the sample with a chromatography medium, and any combination thereof.

### II. Methods of Detecting Surgery-Related Conditions

In various aspects, the modular assays described above may be used in a method for detecting surgery-related conditions within a surgical site of a subject following a surgery. As described above, the surgery-related conditions monitored using the method described herein include, but are not limited to, molecular margin or minimal residual cancer, local immune environment, infection within the surgical site, rejection or failure of a transplant organ, early microvascular free flap failure or necrosis, and any other relevant surgery-related condition without limitation.

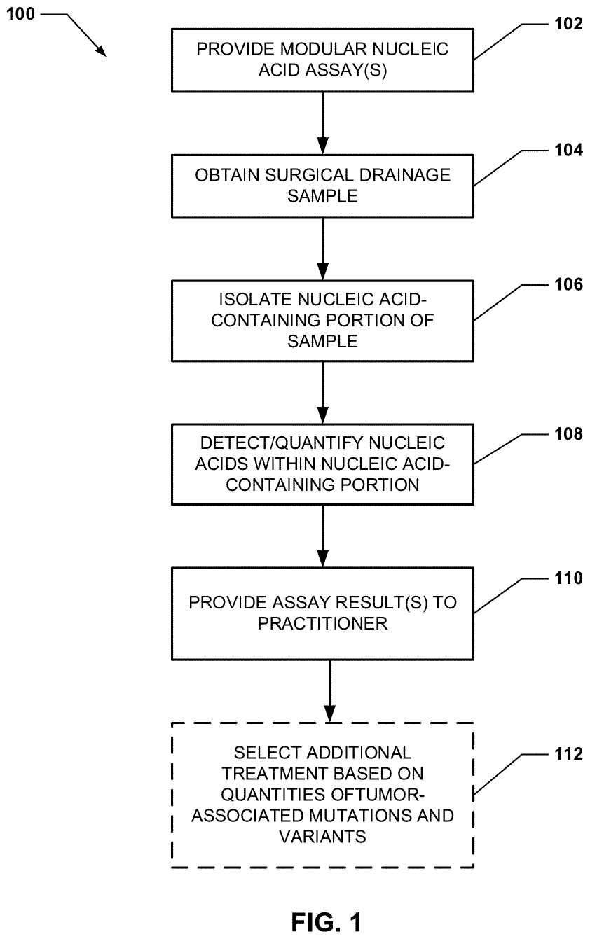

FIG. 1 is a flow chart illustrating the steps of a method 100 for detecting surgery-related conditions in one aspect. The disclosed method includes providing at 102 at least one modular nucleic acid assay from a plurality of modular nucleic acid assays. The at least one modular nucleic acid assay may include at least one or more of the assay modules described above. A practitioner or surgeon may select the at least one modular nucleic acid assay provided in the method as disclosed herein based on the type of surgery, the disorder treated by the surgery, anticipated risk factors such as infection or microvascular failures, anticipated post-surgical treatments, and any other suitable criterion without limitation.

The disclosed method further includes obtaining a surgical drainage sample from the patient at 104. In various aspects, surgical drain fluid samples may be obtained using any suitable surgical drain device associated with any type of surgical procedure without limitation. In some aspects, the surgical drain fluid sample may be obtained using a surgical drain tube, a surgical wound vac, and any other suitable surgical drainage device without limitation. In other aspects, the surgical drain fluid samples may be obtained using custom surgical drain devices specifically provided with elements configured to preserve the integrity of nucleic acids and other analytes within the sample, to perform at least a portion of sample preparation steps, and any other suitable function related to obtaining, preserving, and processing a surgical fluid sample for analysis without limitation.

By way of non-limiting example, the surgical drainage sample may be obtained using a surgical drain configured to collect and preserve nucleic acid biomarkers as described in PCT Application PCT/US2022/020889, the content of which is incorporated herein by reference in its entirety.

In some aspects, a single drain fluid sample may be obtained for analysis by all selected modular assays. In other aspects, separate drain fluid samples may be obtained for each selected modular assay, or separate drain fluid samples may be shared between different subsets of the selected modular assays. In some aspects, the sample may be obtained within about 24 hours of the completion of the surgery, providing the practitioner with timely information regarding genetic material and/or other analytes in the surgical drainage that may be used to select additional treatments. In various aspects, the sample may be obtained within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 24 hours, about 32 hours, about 36 hours, about 48 hours, or about 72 hours of the completion of the surgery.

The disclosed method further includes isolating a portion of the drain fluid sample containing nucleic acids and other analytes described above at 106 for analysis using the selected modular assays. In various aspects, the isolation of the nucleic acids and other analytes is performed in a manner that preserves sufficient integrity of these compounds for detection by the modular assays. The portion of the drain fluid sample containing nucleic acids and other analytes may be isolated from the sample using any suitable method without limitation. Non-limiting examples of suitable isolation methods include filtering the sample, centrifuging the sample, contacting the sample with a chromatography medium, and any combination thereof.

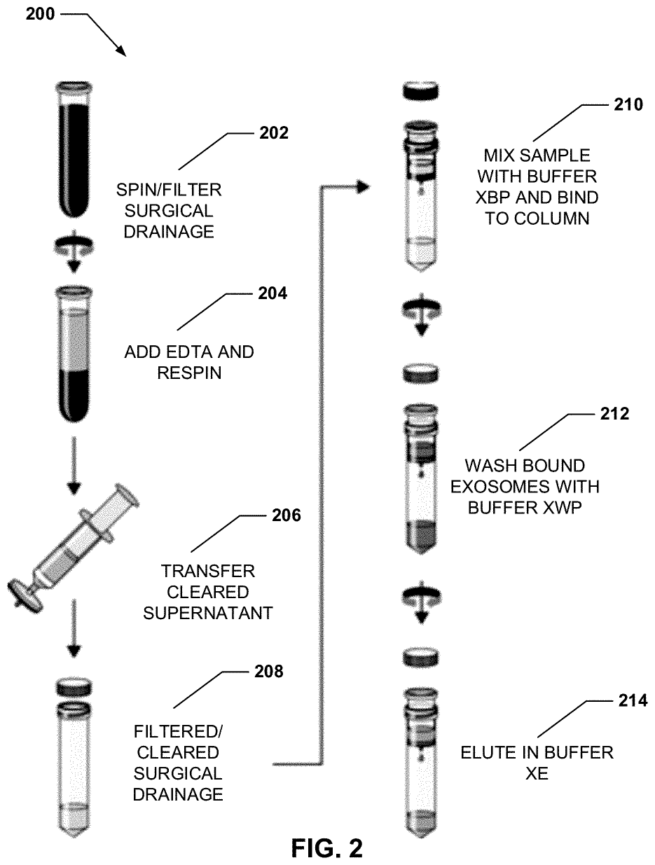

By way of non-limiting example, FIG. 2 is a schematic illustration of a method 200 of isolating the tumor-associated genetic material from the sample in one aspect. As illustrated in FIG. 2, the surgical drainage is centrifuged and filtered at 202. EDTA is added to the sample to inhibit nucleases in the sample and the sample with added EDTA is further centrifuged at 204. The supernatant is removed from the surgical drainage mixture at 206 and retained. In some aspects, the supernatant may be used as-is for the detection and quantification of cell-free DNA, RNA, and proteins. In other aspects, the supernatant may be filtered and cleared at 208 prior to further treatment as illustrated in FIG. 2.

In other aspects, illustrated in FIG. 2, exosomes may be isolated from the supernatant by contacting the supernatant with chromatographic media followed by elution. As illustrated in FIG. 2, the filtered and cleared sample from 208 may be mixed with buffer XBP and bound to a column at 210. The column-bound exosomes may be washed with buffer WP at 212 and eluted from the column with buffer XE at 214. In various aspects, the isolation of the tumor-associated genetic material using the method illustrated in FIG. 2 I configured to yield intact exosomes.

Referring again to FIG. 1, the method 200 further includes detecting and quantifying nucleic acids and other analytes within the isolated portion of the drain fluid sample at 108. Any suitable method may be used to sequence, detect, and quantify the tumor-associated mutations and/or variants including, but not limited to, next generation DNA sequencing, next generation RNA sequencing, next generation protein sequencing, PCR, Western blot, and any combination thereof. In some aspects, a targeted sequence assay panel may be used.

Referring again to FIG. 1, the method may further include providing the assay results including, but not limited to, quantities of detected nucleic acid mutations, variants, and/or over/underexpressions to a practitioner at 110. In some aspects, the assay results may include individual quantities and/or expression profiles of the nucleic acids of interest. In other aspects, assay results may be provided in the form of one or more summary scores obtained by comparing the characteristics of the detected nucleic acids or other analytes to previously-obtained criteria indicative of a surgery-related condition, a prognosis, and/or a recommendation for a post-surgical treatment as described above. In some aspects, an individual summary score may be reported for each selected assay module. In other aspects, the results of two or more assay modules may be combined to produce an overall summary score. In these other aspects, the overall summary score may be based on a mathematic combination of the results of the two or more assay modules including, but not limited to, sums, differences, products, ratios, minima, maxima, means/averages, correlation coefficients, any other suitable mathematical relationship, and any combination thereof.

In various aspects, based on the detected quantities or results of the selected modular assays, the practitioner or surgeon may make a determination of a post-surgical condition or prognosis in the patient, and/or select an additional or follow-up treatment at 112. Non-limiting examples of suitable follow-up treatments include radiotherapy, chemotherapy, follow-up surgery, active surveillance with imaging, antibiotic treatment, and any combination thereof.

In various aspects, the disclosed method provides for the sequencing or molecular measures of a variety of surgery-related conditions including, but not limited to, residual cancer, infection, immune environment, and risk of poor wound healing, using the analysis of surgical drain fluid by interchangeable and recombinable modular assays selected by a practitioner or surgeon.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

What is claimed is:

1. A method for detecting at least one surgery-related condition within a surgical site of a subject following a surgery, the method comprising:
a. providing at least one modular nucleic acid assay from a plurality of modular nucleic acid assays;
b. obtaining a sample from the subject, the sample comprising a surgical drainage sample from the surgical site;
c. isolating a nucleic acid-containing portion from the sample;
d. detecting and quantifying at least a portion of the amount of nucleic acids within the nucleic acid-containing portion using the at least one nucleic acid assay to produce at least one assay result, wherein the at least one assay result is indicative of the at least one surgery-related condition in the subject; and
e. providing the at least one assay result to a practitioner.

2. The method of claim 1, wherein the surgery is selected from a resectioning surgery, a dissection surgery, an excision surgery, a transplant surgery, a reconstructive surgery, and any combination thereof.

3. The method of claim 1, wherein the nucleic acid-containing portion isolated from the sample comprises cfDNA, RNA, exosomes, tumor cells, immune cells, bacterial nucleic acids, viral nucleic acids, and any combination thereof.

4. The method of claim 1, wherein the plurality of modular nucleic acid assays comprise whole genome sequencing, next generation DNA sequencing, next generation RNA sequencing, PCR, Western blot targeted capture, multiplex PCR, methylation & 16S droplet PCR, and any combination thereof.

5. The method of claim 1, wherein each modular nucleic acid assay of the plurality of modular nucleic acid assays is selected from:
a. a surgical margin assay module configured to detect and quantify tumor-associated nucleic acids indicative of minimal residual cancer within the surgical site;
b. an immune environment assay module configured to detect and quantify immune response-related nucleic acids indicative of a local immune environment within the surgical site;
c. an infection assay module configured to detect and quantify infection-associated nucleic acids indicative of an infection within the surgical site;
d. a transplant assay configured to detect and quantify transplant organ nucleic acids indicative of a rejection of a transplant organ by the subject; and
e. a reconstruction assay module configured to detect and quantify flap failure-related nucleic acids indicative of early microvascular free flap failure, necrosis, and any combination thereof

6. The method of claim 1, wherein the plurality of modular nucleic acid assays comprises a combination of at least two modular nucleic acid assays.

7. The method of claim 1, wherein isolating the nucleic acid-containing portion from the sample comprises filtering the sample, centrifuging the sample, contacting the sample with a chromatography medium, and any combination thereof

8. The method of claim 1, further comprising selecting an additional treatment based on the at least one assay result.

9. The method of claim 8, wherein the additional treatment is selected from radiotherapy, chemotherapy, follow-up surgery, active surveillance with imaging, antibiotic therapy, antiviral therapy, and any combination thereof.

10. The method of claim 1, wherein obtaining the sample from the subject further comprises capturing a surgical drainage from a drainage tube associated with the surgery.

11. The method of claim 1, wherein obtaining the sample from the subject further comprises capturing a surgical drainage from the drainage tube within about 24 hours of the surgery.

12. A method of selecting at least one additional treatment for a subject following a surgery, the method comprising:
a. providing at least one modular nucleic acid assay from a plurality of modular nucleic acid assays;
b. obtaining a sample from the subject, the sample comprising a surgical drainage sample from the surgical site;
c. isolating a nucleic acid-containing portion from the sample;
d. detecting and quantifying at least a portion of the amount of nucleic acids within the nucleic acid-containing portion using the at least one nucleic acid assay to produce at least one assay result, wherein the at least one assay result is indicative of the at least one surgery-related condition in the subject; and
e. selecting the at least one additional treatment based on the at least one at least one assay result.

13. The method of claim 12, wherein the at least one additional treatment is selected from radiotherapy, chemotherapy, follow-up surgery, active surveillance with imaging, antibiotic therapy, antifungal therapy, and any combination thereof.

14. The method of claim 12, wherein the nucleic acid-containing portion isolated from the sample comprises cfDNA, RNA, exosomes, tumor cells, immune cells, bacterial nucleic acids, viral nucleic acids, and any combination thereof.

15. The method of claim 12, wherein the plurality of modular nucleic acid assays comprise whole genome sequencing, next generation DNA sequencing, next generation RNA sequencing, PCR, Western blot targeted capture, multiplex PCR, methylation & 16S droplet PCR, and any combination thereof.

16. The method of claim 12, wherein each modular nucleic acid assay of the plurality of modular nucleic acid assays is selected from:
a. a surgical margin assay module configured to detect and quantify tumor-associated nucleic acids indicative of minimal residual cancer within the surgical site;
b. an immune environment assay module configured to detect and quantify immune response-related nucleic acids indicative of a local immune environment within the surgical site;
c. an infection assay module configured to detect and quantify infection-associated nucleic acids indicative of an infection within the surgical site;
d. a transplant assay configured to detect and quantify transplant organ nucleic acids indicative of a rejection of a transplant organ by the subject; and
e. a reconstruction assay module configured to detect and quantify flap failure-related nucleic acids indicative of early microvascular free flap failure, necrosis, and any combination thereof

17. The method of claim 12, wherein the plurality of modular nucleic acid assays comprises a combination of at least two modular nucleic acid assays.

18. The method of claim 12, wherein isolating the nucleic acid-containing portion from the sample comprises filtering the sample, centrifuging the sample, contacting the sample with a chromatography medium, and any combination thereof.

19. The method of claim 12, wherein obtaining the sample from the subject further comprises capturing a surgical drainage from a drainage tube associated with the surgery.
